(19) United States
(12) Patent Application Publication
(10) Pub. No.: US 2007/0243112 A1
(43) Pub. Date: Oct. 18, 2007

(54) Deodorizing Filter

(75) Inventors: Yasutaro Seto, Osaka (JP); Tatsuo Nakamura, Nara (JP); Yoshiharu Nishino, Nara (JP); Naomi Fukui, Osaka (JP)

Correspondence Address: KRATZ, QUINTOS & HANSON, LLP, 1420 K Street, N.W., Suite 400, WASHINGTON, DC, 20005, US

(73) Assignee: SUMINOE TEXTILE CO., LTD., OSAKA-SHI (JP)

(21) Appl. No.: 10/576,264

(22) PCT Filed: Oct. 20, 2004
(86) PCT No.: PCT/JP04/15509

§ 371 Date: May 9, 2007

(30) Foreign Application Priority Data
Oct. 20, 2003 (JP) 2003-358642

(51) Int. Cl. A61L 9/014

(52) U.S. Cl. 422/101

(57) ABSTRACT

A deodorizing filter (1) characterized by comprising a first deodorizing filter (2) regulated so as to have a high-pH environment and a second deodorizing filter (3) regulated so as to have a low-pH environment. The first deodorizing filter and/or second deodorizing filter preferably comprises an active-carbon-filled paper and, supported thereon, a cobalt phthalocyanine complex and an iron phthalocyanine complex.

### FIELD OF THE INVENTION

The present invention relates to a deodorizing filter, for example, for use as a filter material for air conditioner, air cleaner, and refrigerator for home or business, a filter material for removal of odd odor in automobile, or a filter material for removal of foul odor in bath room, and in particular to a filter for decomposing and removing multiple foul odor gases different in properties efficiently.

### BACKGROUND ART

Deodorizing filters have been used in various applications, and the deodorizing methods are grossly grouped into absorptive methods of using an absorbent such as active-carbon or zeolite, catalytic methods of decomposing foul odor substances, for example, by ozone, a photocatalyst, or a metal phthalocyanine complex, and methods in combination of the adsorptive and catalytic methods. Methods of using the superior adsorptive action of active-carbon are well known; but, although these methods are superior in absorbing foul odor components and reducing the odor concentration in the surrounding for a short period of time, they are said to be deodorizing methods that are effective only for a limited period, because the foul odor components are not decomposed; and thus recently, the catalyst and combined methods that decompose and remove the foul odor substances are used more frequently.

For example, Patent Document 1 discloses a composite photocatalyst-containing corrugated filter consisting of a planer sheet (liner) and a corrugated sheet (internal core) in which one member is a photocatalyst-containing sheet and the other member is a special active-carbon-fiber sheet. It also discloses a method of decomposing and removing gases such as acetaldehyde, ammonia, hydrogen sulfide, and acetic acid efficiency by using at least one special active-carbon-fiber sheet selected from a lower aldehydes-removing active-carbon-fiber sheet, an alkaline gas-removing active-carbon-fiber sheet, and an acidic gas-removing active-carbon-fiber sheet, as the special active-carbon-fiber sheet.

Alternatively, Patent Document 2 discloses a deodorizing filter consisting of a first deodorant having a carrier containing a nonwoven ceramic-fiber fabric as its skeleton and a metal oxide containing copper and manganese as the principal components, and a second deodorant having nonwoven ceramic-fiber fabric as the skeleton and a porous zeolite material containing metal oxides of gold and iron. In the deodorizing filter, hydrogen sulfide, mercaptan, and others among the foul odor components are deodorized by the first deodorant having a carrier containing a nonwoven ceramic-fiber fabric as its skeleton and a metal oxide containing copper and manganese as the principal components, and nitrogen-containing compounds such as ammonia and amines are deodorized efficiently as absorbed on the surface of the second deodorant of the zeolite porous material containing the metal oxides of gold and iron.
Patent Document 1: Japanese Unexamined Patent Publication No. 2003-62413
Patent Document 2: Japanese Unexamined Patent Publication No. 6-285144

### SUMMARY OF THE INVENTION

### Problems to be Solved by the Invention

However, these conventional methods, all in combination of the absorptive and catalytic deodorizing methods of decomposing foul odors absorbed on an absorbent catalytically, had a problem that such a system is significantly expensive, although they are useful as the efficient deodorizing methods. Thus, there exists a need for a filter more cost-effective and greater in the deodorizing capacity.

An object of the present invention, which was made under the technical background above, is to provide a filter that is lower in cost but still can decompose and remove basic and acidic odors efficiently at the same time.

### Means for Solving the Problems

To achieve the object, the present invention provides the following means.

[1] A deodorizing filter comprising a first deodorizing filter under high-pH environment and a second deodorizing filter under low-pH environment.

[2] The deodorizing filter as recited in the aforementioned item [1], wherein the first deodorizing filter and the second deodorizing filter are filters of a metal phthalocyanine complex supported on an active-carbon-filled paper.

[3] The deodorizing filter as recited in the aforementioned item [1], wherein the first deodorizing filter and the second deodorizing filter are filters of a cobalt phthalocyanine complex supported on an active-carbon-filled paper.

[4] The deodorizing filter as recited in any one of the aforementioned items [1] to [3], wherein the first deodorizing filter and/or the second deodorizing filter are filters of a cobalt phthalocyanine complex and an iron phthalocyanine complex supported on an active-carbon-filled paper.

[5] The deodorizing filter as recited in any one of the aforementioned items [1] to [3], wherein the first deodorizing filter is a filter of a cobalt phthalocyanine complex and an iron phthalocyanine complex supported on an active-carbon-filled paper.

[6] The deodorizing filter as recited in the aforementioned item [1], wherein the first deodorizing filter and the second deodorizing filter are filters of a cobalt phthalocyanine complex and an iron phthalocyanine complex supported on an active-carbon-filled paper.

[7] The deodorizing filter as recited in any one of the aforementioned items [4] to [6], wherein the weight ratio of the complexes supported, cobalt phthalocyanine complex/iron phthalocyanine complex, is 98/2 to 55/45.

[8] The deodorizing filter as recited in any one of the aforementioned items [4] to [6], wherein the weight ratio of the complexes supported, cobalt phthalocyanine complex/iron phthalocyanine complex, is 95/5 to 85/15.

[9] The deodorizing filter as recited in any one of the aforementioned items [1] to [8], wherein the pH of the high-pH environment is 7.5 to 12.0, and the pH of the low high-pH environment is 1.5 to 5.0.

[10] The deodorizing filter as recited in any one of the aforementioned items [2] to [9], wherein the amount of the complexes supported is in the range of 200 to 20,000 μg with respect to 1 g of the active-carbon-filled paper.

[11] The deodorizing filter as recited in any one of the aforementioned items [2] to [10], wherein the active-carbon-filled paper contains active-carbon at a content of 40 to 80 mass %.

### EFFECTS OF THE INVENTION

Although the mechanism of the invention [1] is yet to be understood sufficiently, it seems that when the first deodorizing filter under high-pH environment and the second deodorizing filter under low-pH environment are combined, basic foul odors such as ammonia and amines are selectively absorbed and efficiently decomposed and removed by the second deodorizing filter under low-pH environment and acidic foul odors such as hydrogen sulfide and methyl mercaptan by the first deodorizing filter under high-pH environment respectively.

For example, in a filter unit having a structure in which a gas passes through the first and second deodorizing filters in that order, acidic gases are first absorbed and decomposed by the first deodorizing filter under high-pH environment. Basic gases pass through the first deodorizing filter under high-pH environment and are absorbed and decomposed by the second deodorizing filter under low-pH environment. Thus, acidic and basic gases are deodorized efficiently, as they are respectively, selectively absorbed by the first and second deodorizing filters. When both the first and second deodorizing filters have a honeycomb structure, the gases are adsorbed and decomposed more efficiently therein than in a single filter, because superposition of the first and second deodorizing filters causes some deviation between the openings of the honeycomb thereof, resulting in turbulence of the foul odor gas stream.

In the invention [2], the deodorizing filter, an active-carbon-filled paper carrying a metal phthalocyanine complex, deodorizes the odor by absorption on active-carbon by its strong absorptive force and decomposition by the oxidation force of the metal phthalocyanine complex. The metal phthalocyanine complex is very effective as a deodorant, because it does not damage the carrier like a photocatalyst and can be deposited on the active-carbon-filled paper without use of a binder resin.

In the invention [3], the deodorizing filter, an active-carbon-filled paper carrying a cobalt phthalocyanine complex, improves the deodorizing capacity, compared to the deodorizing filter using another metal phthalocyanine complex.

In the inventions [4], [5], and [6], the filters, which contain both a cobalt phthalocyanine complex and an iron phthalocyanine complex, improve the odor-removing rate especially of dimethyl sulfide and dimethyl disulfide significantly, because of the synergistic effect by the two complexes.

In particular, in the inventions [5] and [6], the odor-removing rates of dimethyl sulfide and dimethyl disulfide are improved particularly drastically, because the first deodorizing filter carries both a cobalt phthalocyanine complex and an iron phthalocyanine complex.

In the invention [7], in which the weight ratio of the two complexes supported, cobalt phthalocyanine complex/iron phthalocyanine complex, is set to 98/2 to 55/45, it is possible to improve the capacity of the filter deodorizing dimethyl sulfide and dimethyl disulfide further because of the synergistic effect of the two complexes.

In the invention [8], in which the weight ratio of the two complexes supported, cobalt phthalocyanine complex/iron phthalocyanine complex, is set to 95/5 to 85/15, it is possible to improve the capacity of the filter deodorizing dimethyl sulfide and dimethyl disulfide further because of the synergistic effect of the two complexes.

In the invention [9], in which the pH of the high-pH environment is 7.5 to 12.0 and the pH of the low-pH environment is 1.5 to 5.0, the acidic and basic gases are absorbed respectively, selectively on the first and second deodorizing filters, resulting in sufficient deodorizing effect.

In the invention [10], it is possible to improve the deodorizing capacity further, because the amount of the complexes supported is set in the range of 200 to 20,000 μg with respect to 1 g of the active-carbon-filled paper.

In the invention [11], it is possible to obtain a sufficiently high adsorption effect and improve the deodorizing capacity further, because the content of active-carbon in the active-carbon-filled paper is 40 to 80 mass %.

### BRIEF DESCRIPTION OF THE DRAWINGS

FIG. 1 is a perspective view illustrating an embodiment of the deodorizing filter according to the present invention.

### DESCRIPTION OF REFERENCE NUMERALS

1: Deodorizing filter
2: First deodorizing filter
3: Second deodorizing filter

### BEST MODE OF CARRYING OUT THE INVENTION

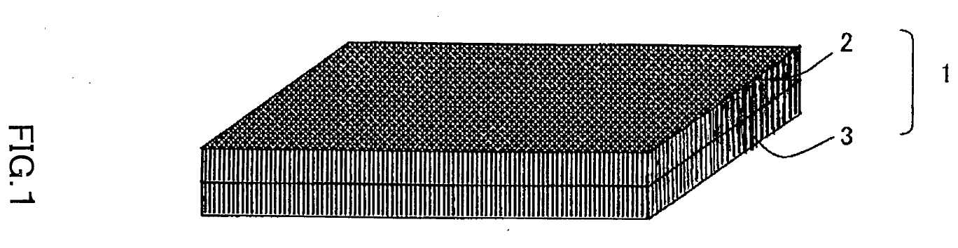

The deodorizing filter according to the present invention will be described with reference to drawings. FIG. 1 is a perspective view illustrating an embodiment of the deodorizing filter according to the present invention. In FIG. 1, a first deodorizing filter under high-pH environment (2) and a second deodorizing filter under low-pH environment (3) are adhered to form a deodorizing filter (1). It is possible to eliminate odor gas, by installing the deodorizing filter (1), for example before or after a fan, and feeding the odor gas into the deodorizing filter (1).

The deodorizing filter (1) according to the present invention has a first deodorizing filter (2) placed under high-pH environment and a second deodorizing filter (3) placed under low-pH environment, but both filters (2) and (3) are preferably an active-carbon-filled paper carrying a metal phthalocyanine complex.

The active-carbon-filled paper can be prepared by a common wet paper-making method. For example, active-carbon and a natural pulp are mixed in water, to give an aqueous slurry. The slurry is adjusted to a particular solid matter concentration while stirred; a cationic or anionic polymer is added thereto; the aqueous aggregate dispersion obtained is sheeted in a paper machine by a wet paper making method; and the sheet thus obtained is then dried, to give an active-carbon-filled paper. The active-carbon-filled paper is then processed, for example, into a filter having a honeycomb shape by using a corrugating machine. The honeycomb filter of the active-carbon-filled paper plays a role as an absorbent for foul odor gases, by the strong absorptive force of active-carbon.

The active-carbon for use in the present invention is preferably an active-carbon-based porous carbon such as coconut shell active-carbon, petroleum pitch-based spherical active-carbon, active-carbon fiber, or wood-based active-carbon, because of its very high adsorptive specific surface area. Among them, use of coconut shell active-carbon is particularly preferable.

Alternatively, the fiber for use in the active-carbon-filled paper is preferably a fibrillated fiber such as of natural pulp, polyolefin, and acryl fiber, and a natural pulp is particularly preferable, from the point of the capacity for adsorbing the metal phthalocyanine complex.

The metal phthalocyanine complex for use in the deodorizing filter (1) according to the present invention is not particularly limited, but, for example, an iron phthalocyanine complex, a cobalt phthalocyanine complex, or the like. Among them, use of a cobalt phthalocyanine complex is preferable, and in such a case, the complex has an advantage in its deodorizing capacity especially for deodorizing methyl mercaptan and acetic acid. The cobalt phthalocyanine complex is not particularly limited, and examples thereof include cobalt phthalocyaninepolysulfonic acid sodium, cobalt phthalocyanineoctacarboxylic acid, cobalt phthalocyaninetetracarboxylic acid, monoaminotricarboxy cobalt phthalocyanine, and the like. The iron phthalocyanine complex is not particularly limited, and examples thereof include iron phthalocyaninetetracarboxylic acid, iron phthalocyanineoctacarboxylic acid, and the like.

In a still more preferable embodiment, the metal phthalocyanine complex is a combination of a cobalt phthalocyanine complex and an iron phthalocyanine complex. Specifically, the first deodorizing filter (2) and/or the second deodorizing filter (3) are particularly preferably an active-carbon-filled paper carrying a cobalt phthalocyanine complex and an iron phthalocyanine complex. Advantageously in such a case, it is possible to improve the odor-removing rate especially of dimethyl sulfide and dimethyl disulfide drastically by synergistic effect of the two complexes. In particular, the cobalt and iron phthalocyanine complexes are favorably supported at least by the first deodorizing filter (2), and in such a case, it is possible to improve the odor-removing rate of dimethyl sulfide and dimethyl disulfide additionally.

The weight ratio of the two complexes, cobalt phthalocyanine complex/iron phthalocyanine complex, is preferably set to 98/2 to 55/45. In such a ratio range, it is possible to raise the capacity for deodorizing dimethyl sulfide and dimethyl disulfide further, by sufficient synergistic effect of the two complexes. It is almost not possible to obtain the synergistic effect, when the ratio is outside the range above. In particular, the weight ratio of the two complexes, cobalt phthalocyanine complex/iron phthalocyanine complex, is particular preferably set to 95/5 to 85/15.

The active-carbon-filled paper is preferably cationized before the metal phthalocyanine complex is deposited on the active-carbon-filled paper. It is a treatment aimed at increasing the amount of the metal phthalocyanine complexes supported, and the cationizing treatment may by any treatment, if it can introduce a cationic group in the chemical structure of the active-carbon-filled paper. In particular, the cationizing treatment by using a quaternary ammonium salt is preferable, and in such a case, it is possible to further increase the amount of the metal phthalocyanine complexes supported. Examples of the quaternary ammonium salts include 3-chloro-2-hydroxypropyltrimethylammonium chloride, glycidyltrimethylammonium chloride, condensation polymers of 3-chloro-2-hydroxypropyltrimethylammonium chloride, and the like.

It is possible to prepare the first deodorizing filter (2) under high-pH environment, by washing and drying the filter (honeycomb filter, etc.) of the cationized active-carbon-filled paper, immersing it in an aqueous alkaline solution containing a metal phthalocyanine complex, and washing and drying the resulting filter. The first deodorizing filter (2) under high-pH environment is not particularly limited to the filter prepared by the method above.

Alternatively, it is possible to prepare the second deodorizing filter (3) under low-pH environment, by immersing the first deodorizing filter (2) under high-pH environment in an acidic aqueous solution and washing and drying the filter. The second deodorizing filter (3) under low-pH environment is not particularly limited to the filter prepared by the method above.

Thus, it is possible to prepare the deodorizing filter (1) according to the present invention, for example, by adhering two kinds of filters, the first deodorizing filter (2) under high-pH environment and the second deodorizing filter (3) under low-pH environment, to each other with an adhesive. It is of course possible to employ a configuration of the first deodorizing filter (2) and the second deodorizing filter (3) being superposed simply (without use of an adhesive). Alternatively, the deodorizing filter (1) may have a configuration in which the first deodorizing filter (2) and the second deodorizing filter (3) are placed as separated from each other. In addition, the number and the combination of the first deodorizing filters (2) and the second deodorizing filters (3) may be altered as needed according to the kind and concentration of the gas to be removed.

In addition, the shape of the first deodorizing filter (2) or the second deodorizing filter (3) is not particularly limited. For example, it may be formed into a planar sheet, a corrugated sheet, or a sheet in the honeycomb structure. In short, the shape is arbitrary, if the two deodorizing filters (2) and (3) allow passage of the gas to be removed.

The acidic aqueous solution above is not particularly limited, and, for example, a nonvolatile mineral acid such as aqueous phosphoric acid solution, and the like.

The aqueous alkaline solution is also not particularly limited, and is, for example, an aqueous sodium hydroxide solution or the like.

The adhesive is also not particularly limited, and, for example, an aqueous emulsion such as of an ethylene-vinyl acetate copolymer or an acrylic acid polymers, a hot melt resin, or the like.

In the present invention, the high-pH environment is preferably at a pH in the range of 7.5 to 12.0. A high-pH environment at a pH of less than 7.5 is unfavorable, because it deteriorates the speed of absorbing acidic odors. A high-pH environment at a pH of more than 12.0 is also unfavorable, because it deteriorates the stability of the metal phthalocyanine complex. In particular, the high-pH environment is particularly preferably at a pH in the range of 8.0 to 11.0.

Alternatively, the low-pH environment is preferably at a pH in the range of 1.5 to 5.0. A low-pH environment at a pH of less than 1.5 is unfavorable, because the cellulose in the active-carbon-filled paper is hydrolyzed, resulting in easier separation of active-carbon. Alternatively, a low-pH environment at a pH of more than 5.0 is also unfavorable, because it leads to deterioration in the speed of absorbing basic odors. In particular, the low-pH environment is particularly preferably at a pH in the range of 2.0 to 4.0.

In the present invention, the amount of the metal phthalocyanine complex supported is in the range of 200 to 20,000 pg with respect to 1 g of the active-carbon-filled paper. An amount of less than 200 μg is unfavorable because of drastic decrease in decomposition speed. Alternatively, an amount of more than 20,000 μg leads to saturation of the deodorizing capacity and only to increase in cost, and is thus unfavorable. In particular, the amount of the metal phthalocyanine complex supported is particularly preferably in the range of 300 to 3,000 μg with respect to 1 g of the active-carbon-filled paper.

Alternatively, the content of active-carbon in the active-carbon-filled paper is preferably 40 to 80 mass %. A content of less than 40 mass % is unfavorable, because of decrease in foul odor gas absorption speed. Alternatively, a content of more than 80 mass % inevitably results in decrease in cellulosic fiber content and thus deterioration in the physical strength of the filter, and thus is unfavorable. In particular, the content of active-carbon in the active-carbon-filled paper is more preferably 55 to 75 mass %.

In the present invention, it is also possible to employ a configuration in which the active-carbon-filled paper contains another deodorant, an odor absorbent, an additive, or the like. Examples of the other deodorants include hydrazine derivatives and polyvinylamine compounds. Examples of the odor absorbents include, in addition to the active-carbon, porous inorganic materials such as zeolite.

Examples of the hydrazine derivatives include reaction products of a hydrazine compound and a long-chain aliphatic compound, reaction products of a hydrazine compound and an aromatic compound, and the like. Particularly favorable are reaction products of one or more compounds selected from the group consisting of hydrazines and semicarbazides, one or more compounds selected from the group consisting of monocarboxylic acids, dicarboxylic acids, aromatic monocarboxylic acids and aromatic dicarboxylic acids having 8 to 16 carbon atoms, and one or more compounds selected from the group consisting of monoglycidyl derivatives and diglycidyl derivatives having 8 to 16 carbon atoms. Use of such a hydrazine derivative is effective in improving the deodorizing capacity additionally. Typical examples of the reaction products include, but are not particularly limited to, sebacic dihydrazide, dodecanedioic acid dihydrazide, isophthalic dihydrazide, and the like.

### EXAMPLES

Hereinafter, typical Examples of the present invention will be described.

### Example 1

70 parts by mass of coconut shell active-carbon and 30 parts by mass of a natural pulp were added into 200 parts by mass of water, to give an aqueous slurry. The aqueous aggregate dispersion obtained was sheeted in a paper machine by a wet paper making method, and the sheet was dried, to give an active-carbon-filled paper. Part of the active-carbon-filled papers obtained was processed into corrugated paper in a corrugating machine. The corrugated paper was adhered to a planar paper with an adhesive of an ethylene-vinyl acetate copolymer, to give a filter material having a cell density of 230 cell/inch². The filter material was cationized in an aqueous 3-chloro-2-hydroxypropyltrimethylammonium chloride solution, and then dried. The cationized filter material was further immersed in an aqueous alkaline solution containing 0.5 mass % cobalt phthalocyaninepolysulfonic acid sodium and 5 g/L sodium hydroxide, washed with water, and then dried, to give a first deodorizing filter (2) under a high-pH environment (pH 10.0).

Then, half of the first deodorizing filters were immersed in an aqueous 2 mass % phosphate solution, washed with water, and dried, to give a second deodorizing filter (3) under a low-pH environment (pH 3.0). The first deodorizing filter (2) and the second deodorizing filter (3) were adhered to each other with an adhesive of an ethylene-vinyl acetate copolymer, to give a deodorizing filter (1). The amount of the cobalt phthalocyaninepolysulfonic acid sodium supported on the active-carbon-filled paper in the deodorizing filter obtained was 400 μg/g. The content of coconut shell active-carbon in the active-carbon-filled paper was 70 mass %.

### Example 2

A deodorizing filter (1) was prepared in a similar manner to Example 1, except that an aqueous alkaline solution containing 0.5 mass % cobalt phthalocyaninepolysulfonic acid sodium and 50 g/L sodium hydroxide was used as the aqueous alkaline solution. The pH of the first deodorizing filter (2) under high-pH environment was 12.0.

### Example 3

A deodorizing filter (1) was prepared in a similar manner to Example 1, except that an aqueous alkaline solution containing 1.5 mass % cobalt phthalocyaninepolysulfonic acid sodium and 5 g/L sodium hydroxide was used as the aqueous alkaline solution. The amount of the cobalt phthalocyaninepolysulfonic acid sodium supported on the active-carbon-filled paper in the deodorizing filter obtained was 1,000 μg/g.

### Example 4

A deodorizing filter (1) was prepared in a similar manner to Example 1, except that 30 parts by mass of coconut shell active-carbon and 30 parts by mass of a natural pulp were added into 200 parts by mass of water to give an aqueous slurry. The content of coconut shell active-carbon in the active-carbon-filled paper was 50 mass %.

### Example 5

A deodorizing filter (1) was prepared in a similar manner to Example 1, except that an aqueous 5 mass % phosphate solution was used as the aqueous phosphate solution. The pH of the second deodorizing filter (3) under low-pH environment was 1.5.

### Example 6

A deodorizing filter (1) was prepared by inserting the second deodorizing filter (3) obtained in Example 1 between two first deodorizing filters (2) (2) obtained in Example 1 and making them adhere to each other.

### Example 7

A deodorizing filter (1) was prepared by superposing two first deodorizing filters (2) obtained in Example 1 on two second deodorizing filter (3) obtained in Example 1 and making them adhere to each other.

### Example 8

A deodorizing filter (1) was prepared in a similar manner to Example 1, except that an aqueous alkaline solution containing 0.485 mass % cobalt phthalocyaninepolysulfonic acid sodium, 0.015 mass % iron phthalocyaninetetracarboxylic acid sodium, and 5 g/L sodium hydroxide was used as the aqueous alkaline solution. The pH of the first deodorizing filter (2) under high-pH environment was 12.0. The amount of the metal phthalocyanine complexes (sum of the cobalt and iron complexes) supported on the active-carbon-filled paper was 400 μg/g. Specifically, the amount of the cobalt phthalocyaninepolysulfonic acid sodium supported on the active-carbon-filled paper was 388 μg/g, and the amount of the iron phthalocyaninetetracarboxylic acid sodium supported on the active-carbon-filled paper was 12 μg/g.

### Example 9

A deodorizing filter (1) was prepared in a similar manner to Example 1, except that an aqueous alkaline solution containing 0.45 mass % cobalt phthalocyaninepolysulfonic acid sodium, 0.05 mass % iron phthalocyaninetetracarboxylic acid sodium, and 5 g/L sodium hydroxide was used as the aqueous alkaline solution. The pH of the first deodorizing filter (2) under high-pH environment was 12.0. The amount of the metal phthalocyanine complexes (sum of the cobalt and iron complexes) supported on the active-carbon-filled paper was 400 μg/g. Specifically, the amount of the cobalt phthalocyaninepolysulfonic acid sodium supported on the active-carbon-filled paper was 360 μg/g, and the amount of the iron phthalocyaninetetracarboxylic acid sodium supported on the active-carbon-filled paper was 40 μg/g.

### Example 10

A deodorizing filter (1) was prepared in a similar manner to Example 1, except that an aqueous alkaline solution containing 0.30 mass % cobalt phthalocyaninepolysulfonic acid sodium, 0.20 mass % iron phthalocyaninetetracarboxylic acid sodium, and 5 g/L sodium hydroxide was used as the aqueous alkaline solution. The pH of the first deodorizing filter (2) under high-pH environment was 12.0. The amount of the metal phthalocyanine complexes (sum of the cobalt and iron complexes) supported on the active-carbon-filled paper was 400 μg/g. Specifically, the amount of the cobalt phthalocyaninepolysulfonic acid sodium supported on the active-carbon-filled paper was 240 μg/g, and the amount of the iron phthalocyaninetetracarboxylic acid sodium supported on the active-carbon-filled paper was 160 μg/g.

### Comparative Example 1

A deodorizing filter (1) was prepared in a similar manner to Example 1, except that an aqueous solution containing 0.5 mass % cobalt phthalocyaninepolysulfonic acid sodium was used as the aqueous alkaline solution. The pH of the first deodorizing filter (2) was 7.0.

### Comparative Example 2

A deodorizing filter (1) was prepared in a similar manner to Example 1, except that 10 parts by mass of coconut shell active-carbon and 30 parts by mass of a natural pulp were added into 200 parts by mass of water to give an aqueous slurry. The content of coconut shell active-carbon in the active-carbon-filled paper was 25 mass %.

### Comparative Example 3

A deodorizing filter (1) was prepared in a similar manner to Example 1, except that an aqueous 0.1 mass % phosphate solution was used as the aqueous phosphate solution. The pH of the second deodorizing filter (3) was 6.0.

### Comparative Example 4

A deodorizing filter (1) was prepared in a similar manner to Example 1, except that an aqueous alkaline solution containing 0.1 mass % cobalt phthalocyaninepolysulfonic acid sodium and 5 g/L sodium hydroxide was used as the aqueous alkaline solution. The amount of the cobalt phthalocyaninepolysulfonic acid sodium supported on the active-carbon-filled paper in the deodorizing filter obtained was 150 μg/g.

### Comparative Example 5

A deodorizing filter (1) was prepared only by using a first deodorizing filter (2) obtained in Example 1.

### Comparative Example 6

A deodorizing filter (1) was prepared only by using two first deodorizing filters (2) (2) obtained in Example 1.

### Comparative Example 7

A deodorizing filter (1) was prepared in a similar manner to Example 1, except that no coconut shell active-carbon was used.

Each of the deodorizing filters thus prepared was evaluated according to the test methods below. The results are summarized in Table 3.

TABLE 1

| | | | Amount of metal | | | |
|---|---|---|---|---|---|---|
| | pH of | pH of | phthalocyanine complex | Content (mass %) | Number of | Number of |
| | first | second | supported on 1 g of | of active-carbon | first | second |
| | deodorizing | deodorizing | active-carbon- | in active-carbon- | deodorizing | deodorizing |
| | filter | filter | filled paper (μg) | filled paper | filters | filters |
| Example1 | 10.0 | 3.0 | 400 | 70 | 1 | 1 |
| Example2 | 12.0 | 3.0 | 400 | 70 | 1 | 1 |
| Example3 | 10.0 | 3.0 | 1000 | 70 | 1 | 1 |
| Example4 | 10.0 | 3.0 | 400 | 50 | 1 | 1 |
| Example5 | 10.0 | 1.5 | 400 | 70 | 1 | 1 |
| Example6 | 10.0 | 3.0 | 400 | 70 | 2 | 1 |
| Example7 | 10.0 | 3.0 | 400 | 70 | 2 | 2 |
| Example8 | 10.0 | 3.0 | 400 (Co complex: 388 μg | 70 | 1 | 1 |
| | | | and Fe complex: 12 μg) | | | |
| Example9 | 10.0 | 3.0 | 400 (Co complex: 360 μg | 70 | 1 | 1 |
| | | | and Fe complex: 40 μg) | | | |
| Example10 | 10.0 | 3.0 | 400 (Co complex: 240 μg | 70 | 1 | 1 |
| | | | and Fe complex: 160 μg) | | | |

TABLE 2

| | pH of | pH of | Amount of complex | Content (mass %) | Number of | Number of |
|---|---|---|---|---|---|---|
| | first | second | supported on 1 g of | of active-carbon | first | second |
| | deodorizing | deodorizing | active-carbon- | in active-carbon- | deodorizing | deodorizing |
| | filter | filter | filled paper(μg) | filled paper | filters | filters |
| Comparative | 7.0 | 3.0 | 400 | 70 | 1 | 1 |
| Example1 | | | | | | |
| Comparative | 10.0 | 3.0 | 400 | 25 | 1 | 1 |
| Example2 | | | | | | |
| Comparative | 10.0 | 6.0 | 400 | 70 | 1 | 1 |
| Example3 | | | | | | |
| Comparative | 10.0 | 3.0 | 150 | 70 | 1 | 1 |
| Example4 | | | | | | |
| Comparative | 10.0 | — | 400 | 70 | 1 | 0 |
| Example5 | | | | | | |
| Comparative | 10.0 | — | 400 | 70 | 2 | 0 |
| Example6 | | | | | | |
| Comparative | 10.0 | 3.0 | 400 | −(0) | 1 | 1 |
| Example7 | | | | | | |

TABLE 3

| | Result of deodorizing capacity test | | | | | | | |
|---|---|---|---|---|---|---|---|---|
| | | | Hydrogen | | Methyl | | | |
| | Ammonia | | sulfide | | mercaptan | | Acetic acid | |
| | Removal | Evaluation | Removal | Evaluation | Removal | Evaluation | Removal | Evaluation |
| | rate | result | rate | result | rate | result | rate | result |
| Example1 | 99 | ⊚ | 98 | ⊚ | 98 | ⊚ | 96 | ⊚ |
| Example2 | 99 | ⊚ | 100 | ⊚ | 100 | ⊚ | 98 | ⊚ |
| Example3 | 100 | ⊚ | 100 | ⊚ | 100 | ⊚ | 98 | ⊚ |
| Example4 | 95 | ⊚ | 93 | ◯ | 93 | ◯ | 92 | ◯ |
| Example5 | 100 | ⊚ | 98 | ⊚ | 98 | ⊚ | 95 | ⊚ |
| Example6 | 99 | ⊚ | 100 | ⊚ | 100 | ⊚ | 100 | ⊚ |
| Example7 | 100 | ⊚ | 100 | ⊚ | 100 | ⊚ | 100 | ⊚ |
| Example8 | 100 | ⊚ | 100 | ⊚ | 100 | ⊚ | 100 | ⊚ |
| Example9 | 100 | ⊚ | 100 | ⊚ | 100 | ⊚ | 100 | ⊚ |
| Example10 | 100 | ⊚ | 100 | ⊚ | 99 | ⊚ | 99 | ⊚ |
| Comparative | 99 | ⊚ | 92 | ◯ | 91 | ◯ | 89 | Δ |
| Example1 | | | | | | | | |
| Comparative | 87 | Δ | 85 | Δ | 79 | X | 63 | X |
| Example2 | | | | | | | | |
| Comparative | 82 | X | 96 | ⊚ | 95 | ⊚ | 95 | ⊚ |
| Example3 | | | | | | | | |
| Comparative | 91 | ◯ | 89 | Δ | 87 | Δ | 85 | Δ |
| Example4 | | | | | | | | |
| Comparative | 38 | X | 81 | X | 79 | X | 75 | X |
| Example5 | | | | | | | | |
| Comparative | 51 | X | 91 | ◯ | 90 | ◯ | 87 | Δ |
| Example6 | | | | | | | | |
| Comparative | 48 | X | 42 | X | 37 | X | 35 | X |
| Example7 | | | | | | | | |
| | Result of deodorizing capacity test | | | | | | | |
| | | | | | Dimethyl | | Dimethyl | |
| | Acetaldehyde | | Formaldehyde | | sulfide | | disulfide | |
| | Removal | Evaluation | Removal | Evaluation | Removal | Evaluation | Removal | Evaluation |
| | rate | result | rate | result | rate | result | rate | result |
| Example1 | 95 | ⊚ | 95 | ⊚ | 85 | Δ | 92 | ◯ |
| Example2 | 97 | ⊚ | 98 | ⊚ | 86 | Δ | 93 | ◯ |
| Example3 | 96 | ⊚ | 96 | ⊚ | 88 | Δ | 95 | ⊚ |
| Example4 | 90 | ◯ | 92 | ◯ | 85 | Δ | 85 | Δ |
| Example5 | 95 | ⊚ | 97 | ⊚ | 95 | ⊚ | 97 | ⊚ |
| Example6 | 99 | ⊚ | 99 | ⊚ | 99 | ⊚ | 99 | ⊚ |
| Example7 | 100 | ⊚ | 100 | ⊚ | 100 | ⊚ | 100 | ⊚ |
| Example8 | 96 | ⊚ | 98 | ⊚ | 93 | ◯ | 98 | ⊚ |
| Example9 | 98 | ⊚ | 99 | ⊚ | 93 | ◯ | 99 | ⊚ |
| Example10 | 98 | ⊚ | 99 | ⊚ | 91 | ◯ | 97 | ⊚ |
| Comparative | 87 | Δ | 89 | Δ | 82 | X | 90 | ◯ |
| Example1 | | | | | | | | |
| Comparative | 61 | X | 68 | X | 47 | X | 60 | X |
| Example2 | | | | | | | | |
| Comparative | 95 | ⊚ | 95 | ⊚ | 84 | X | 90 | ◯ |
| Example3 | | | | | | | | |
| Comparative | 85 | Δ | 87 | Δ | 74 | X | 83 | X |
| Example4 | | | | | | | | |
| Comparative | 72 | X | 78 | X | 57 | X | 68 | X |
| Example5 | | | | | | | | |
| Comparative | 85 | Δ | 87 | Δ | 81 | X | 89 | Δ |
| Example6 | | | | | | | | |
| Comparative | 35 | X | 37 | X | 24 | X | 35 | X |
| Example7 | | | | | | | | |

<Deodorizing Capacity Test>

(Ammonia-Deodorizing Capacity)

A test kit that is equipped with a fan supplying air at a flow rate of 5 liters per minute from one end of the circular tube and contains a circular test piece (diameter: 50 mm, and thickness: 20 mm (composite of two deodorizing filter layers each having a thickness of 10 mm)), which was cut out of a deodorizing filter (1), fixed with a sample holder placed in the middle of the long circular tube was placed in an acrylic box having a capacity of 250 liter; ammonia gas was injected into the box to a concentration of 100 ppm; the concentration of ammonia after 1 hour was determined; and the total amount of the ammonia gas removed was calculated from the observed values. Thus, the ammonia gas removal rate (%) was calculated.

(Hydrogen Sulfide-Deodorizing Capacity)

A hydrogen sulfide removal rate (%) was calculated in a similar manner to the ammonia deodorizing capacity measurement, except that hydrogen sulfide gas was injected instead of ammonia gas into the acrylic box to a concentration of 10 ppm.

(Methyl Mercaptan-Deodorizing Capacity)

A methyl mercaptan gas removal rate (%) was calculated in a similar manner to the ammonia deodorizing capacity measurement, except that methyl mercaptan gas was injected instead of ammonia gas into the acrylic box to a concentration of 10 ppm.

(Acetic Acid-Deodorizing Capacity)

An acetic acid gas removal rate (%) was calculated in a similar manner to the ammonia deodorizing capacity measurement, except that acetic acid gas was injected instead of ammonia gas into the acrylic box to a concentration of 10 ppm.

(Acetaldehyde-Deodorizing Capacity)

An acetaldehyde gas removal rate (%) was calculated in a similar manner to the ammonia deodorizing capacity measurement, except that acetaldehyde gas was injected instead of ammonia gas into the acrylic box to a concentration of 10 ppm.

(Formaldehyde-Deodorizing Capacity)

A formaldehyde gas removal rate (%) was calculated in a similar manner to the ammonia deodorizing capacity measurement, except that formaldehyde gas was injected instead of ammonia gas into the acrylic box to a concentration of 10 ppm.

(Dimethyl Sulfide-Deodorizing Capacity)

A dimethyl sulfide gas removal rate (%) was calculated in a similar manner to the ammonia deodorizing capacity measurement, except that dimethyl sulfide gas was injected instead of ammonia gas into the acrylic box to a concentration of 10 ppm. Dimethyl sulfide has a seashore-like odor.

(Dimethyl Disulfide-Deodorizing Capacity)

A dimethyl disulfide gas removal rate (%) was calculated in a similar manner to the ammonia deodorizing capacity measurement, except that dimethyl disulfide gas was injected instead of ammonia gas into the acrylic box to a concentration of 10 ppm. Dimethyl disulfide has a pickle-like odor.

A removal rate of 95% or more was designated as “\textcircled{⊙}”; a removal rate of 90% or more and less than 95%, “∘”; a removal rate of 85% or more and less than 90%, “Δ”; and a removal rate of less than 85%, “×”.

As apparent from the Table, each of the deodorizing filters obtained in Examples 1 to 7 according to the present invention had a superior deodorizing capacity to all of ammonia, hydrogen sulfide, methyl mercaptan, acetic acid, acetaldehyde, and formaldehyde. In addition, each of the deodorizing filters obtained in Examples 8 to 10 according to the present invention had a superior deodorizing capacity to all of ammonia, hydrogen sulfide, methyl mercaptan, acetic acid, acetaldehyde, and formaldehyde, as well as dimethyl sulfide and dimethyl disulfide.

In contrast, all of the deodorizing filters obtained in Comparative Examples 1 to 7 did not have a sufficiently high deodorizing capacity.

This application claims priority to Japanese Patent Application No. 2003-358642 filed on Oct. 20, 2003, the disclosure of which is incorporated by reference in its entirety.

The terms and description herein are only aimed at describing favorable embodiments of the present invention, and the present invention is not restricted thereby. The present invention permits any design-change, unless it deviates from the soul, if it is within the limits by which the claim was performed.

### INDUSTRIAL APPLICABILITY

The deodorizing filter according to the present invention is used, for example, as a filter material for air conditioner, air cleaner, and refrigerator for home or business, a filter material for removal of odd odor in automobile, a filter material for removal of foul odor in bath room, or the like.

1 A deodorizing filter comprising a first deodorizing filter regulated so as to have a high-pH environment and a second deodorizing filter regulated so as to have a low-pH environment,
wherein the second deodorizing filter are filters of a cobalt phthalocyanine complex and an iron phthalocyanine complex supported on an active-carbon-filled paper.

2 (canceled)

3 (canceled)

4 The deodorizing filter as recited in claim 1, wherein the weight ratio of the complexes supported, cobalt phthalocyanine complex/iron phthalocyanine complex, is 98/2 to 55/45.

5 The deodorizing filter as recited in claim 1, wherein the weight ratio of the complexes supported, cobalt phthalocyanine complex/iron phthalocyanine complex, is 95/5 to 85/15.

6 The deodorizing filter as recited in claim 1, wherein the pH of the high-pH environment is 7.5 to 12.0 and the pH of the low-pH environment is 1.5 to 5.0.

7 The deodorizing filter as recited in claim 1, wherein the amount of the complexes supported is in the range of 200 to 20,000 μg with respect to 1 g of the active-carbon-filled paper.

8 The deodorizing filter as recited in claim 1, wherein the active-carbon-filled paper contains active-carbon at a content of 40 to 80 mass %.

9 (canceled)

10 (canceled)

11 (canceled)

12 A deodorizing filter comprising a first deodorizing filter regulated so as to have a high-pH environment and a second deodorizing filter regulated so as to have a low-pH environment,
wherein the first deodorizing filter is a filter of a cobalt phthalocyanine complex and an iron phthalocyanine complex supported on an active-carbon-filled paper.

13 The deodorizing filter as recited in claim 12, wherein the weight ratio of the complexes supported, cobalt phthalocyanine complex/iron phthalocyanine complex, is 98/2 to 55/45.

14 The deodorizing filter as recited in claim 12, wherein the weight ratio of the complexes supported, cobalt phthalocyanine complex/iron phthalocyanine complex, is 95/5 to 85/15.

15 The deodorizing filter as recited in claim 12, wherein the pH of the high-pH environment is 7.5 to 12.0 and the pH of the low pH environment is 1.5 to 5.0.

16 The deodorizing filter as recited in claim 12, wherein the amount of the complexes supported is in the range of 200 to 20,000 μg with respect to 1 g of the active-carbon-filled paper.

17 The deodorizing filter as recited in claim 12, wherein the active-carbon-filled paper contains active-carbon at a content of 40 to 80 mass %.

18 A deodorizing filter comprising a first deodorizing filter regulated so as to have a high-pH environment and a second deodorizing filter regulated so as to have a low-pH environment,
wherein the first deodorizing filter and the second deodorizing filter are filters of a cobalt phthalocyanine complex and an iron phthalocyanine complex supported on an active-carbon-filled paper.

19 The deodorizing filter as recited in claim 18, wherein the weight ratio of the complexes supported, cobalt phthalocyanine complex/iron phthalocyanine complex, is 98/2 to 55/45.

20 The deodorizing filter as recited in claim 18, wherein the weight ratio of the complexes supported, cobalt phthalocyanine complex/iron phthalocyanine complex, is 95/5 to 85/15.

21 The deodorizing filter as recited in claim 18, wherein the pH of the high-pH environment is 7.5 to 12.0 and the pH of the low pH environment is 1.5 to 5.0.

22 The deodorizing filter as recited in claim 18, wherein the amount of the complexes supported is in the range of 200 to 20,000 μg with respect to 1 g of the active-carbon-filled paper.

23 The deodorizing filter as recited in claim 18, wherein the active-carbon-filled paper contains active-carbon at a content of 40 to 80 mass %.
